(19) United States
(12) Patent Application Publication
(10) Pub. No.: US 2026/0355520 A1
(43) Pub. Date: Nov. 12, 2026

(54) MONITORING, PROGRAMMING, AND CONTROLLING NEURAL STIMULATION THERAPY

(71) Applicant: Saluda Medical Pty Limited, Macquarie Park (AU)

(72) Inventors: Leah Rebecca MULLER, Bloomington, MN (US); Ian Cameron GOULD, Macquarie Park (AU); Thomas John Kennedy PARISH, Macquarie Park (AU); Peter Scott Vallack SINGLE, Macquarie Park (AU); Dean Michael KARANTONIS, Macquarie Park (AU)

(21) Appl. No.: 19/671,816

(22) Filed: May 8, 2026

Related U.S. Application Data
(60) Provisional application No. 63/802,186, filed on May 8, 2025.

(51) Int. Cl. A61N 1/36; A61N 1/02

(52) U.S. Cl. CPC A61N 1/36135; A61N 1/025; A61N 1/3615

(57) ABSTRACT

Disclosed is a method of determining one or more parameters of a model of instantaneous neural accommodation of a neural pathway of a patient. The method comprises delivering a test sequence of neural stimuli to the neural pathway of the patient, each neural stimulus in the test sequence being delivered according to a respective stimulus intensity parameter. For each delivered neural stimulus in the test sequence, capture at least one signal window from a signal sensed on the neural pathway subsequent to the delivery of that neural stimulus, and measure, from the at least one signal window, an intensity of a neural response evoked by that neural stimulus. Determine, from the respective stimulus intensity parameters and the measured intensities of the neural responses evoked by the test sequence, first values for the one or more parameters of the model of instantaneous neural accommodation of the neural pathway.

The present application claims priority from U.S. Provisional Patent Application No. 63/802,186 filed on 8 May 2025, the contents of which are incorporated herein by reference in their entirety.

### TECHNICAL FIELD

The present invention relates to neural stimulation and in particular to methods for monitoring, programming, and controlling closed-loop neural stimulation therapy.

### BACKGROUND OF THE INVENTION

There are a range of situations in which it is desirable to apply neural stimuli in order to alter neural function, a process known as neuromodulation. For example, neuromodulation is used to treat a variety of disorders including chronic pain, movement disorders, and voiding disorders. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either inhibitory or excitatory effects on neural networks. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in an orthodromic direction (in afferent fibres this means towards the head, or rostral) and in an antidromic direction (in afferent fibres this means towards the cauda, or caudal). Action potentials propagating along Aβ (A-beta) fibres being stimulated in this way may inhibit the transmission of pain from a region of the body innervated by the target neural fibres (the dermatome) to the brain. To sustain the pain relief effects, stimuli are applied repeatedly, for example at a stimulus frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In some neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ fibres or recruitment of undesired fibre classes. When recruitment is too large, Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) or postural changes of the implant recipient (patient), either of which can significantly alter the neural recruitment arising from a given stimulus, and therefore the therapeutic range. The spinal cord itself moves within the cerebrospinal fluid (CSF) with respect to the dura and the electrode array. During postural changes, the distance between the spinal cord and the electrode array can change significantly. This effect is so large that postural changes alone can cause a previously comfortable and effective stimulus regime to become either ineffectual or painful.

Attempts have been made to address such problems by way of feedback or closed-loop control, such as using the methods set forth in International Patent Publication No. WO2012/155188 by the present applicant, the content of which is incorporated herein by reference. Feedback control seeks to compensate for relative nerve/electrode movement by controlling the intensity of the delivered stimuli to maintain neural recruitment at or near a target value. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to bring the response intensity closer to the target value.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be sensed by a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the content of which is incorporated herein by reference.

It is generally assumed for the purposes of closed-loop neural stimulation system (CLNS) system design that neural fibres, at least for stimulus frequencies in the tens of Hz range, are “memoryless”, i.e. their evoked response to a stimulus of a given intensity is independent of the history of stimuli preceding the evoking stimulus. However, recent evidence suggests that neural fibres do have some degree of “memory” even over time scales of tens of milliseconds, which are comparable to the stimulation periods of CLNS systems with stimulus frequencies in the tens of Hz. In other words, the intensity of an evoked response depends to some extent on past stimuli as well as on the most recent stimulus. The stimulus frequency at which this “memory” significantly affects response intensity may be related to the time period over which the excitability of the target neural fibres returns to baseline after the target neural fibres have been activated by one or more previous stimuli. This period in turn may be related to the recovery time constants of the ion channels traversing the neural membrane of each fibre. If these time constants are sufficiently long compared to the stimulation period, conventionally designed and programmed CLNS systems may be less effective at maintaining neural recruitment at or near a target value, particularly if the target value is changing with time or there is some kind of disturbance to the feedback loop. This difficulty becomes even greater if the memory-related characteristics of the target fibres change over time, e.g. with disease progression.

Any discussion of documents, acts, materials, devices, articles or the like which has been included in the present Background is solely for the purpose of providing a context for the present technology. It is not to be taken as an admission that any or all of these matters form part of the prior art base or were common general knowledge in the field relevant to the present technology as it existed before the priority date of each claim of the present disclosure.

### SUMMARY OF THE INVENTION

The present invention seeks to provide methods and systems for programming, operating, and monitoring neural stimulation devices, which will overcome or substantially ameliorate at least some of the deficiencies of the prior art, or at least provide an alternative.

Some aspects of the present disclosure relate to a method of determining one or more parameters of a model of instantaneous neural accommodation (INA) of a neural pathway of a patient. The method comprises delivering a test sequence of neural stimuli to the neural pathway of the patient, each neural stimulus in the test sequence being configured to evoke a neural response from the neural pathway, each neural stimulus in the test sequence being delivered according to a respective stimulus intensity parameter. The method further comprises for each delivered neural stimulus in the test sequence, capturing at least one signal window from a signal sensed on the neural pathway subsequent to the delivery of that neural stimulus, and measuring, from the at least one signal window, an intensity of a neural response evoked by that neural stimulus. The method further comprises determining, from (i) the respective stimulus intensity parameters and (ii) the measured intensities of the neural responses evoked by the test sequence, first values for the one or more parameters of the model of instantaneous neural accommodation (INA) of the neural pathway.

In some implementations, determining the first values for the one or more parameters comprises applying a regression model to the respective stimulus intensity parameters and the measured intensities of the neural responses. In some implementations, the model of INA is an autoregressive moving average (ARMA) model.

In some implementations, the test sequence comprises a step of the stimulus intensity parameters. In some implementations, the model of INA is a bifurcated model, and wherein the one or more parameters comprise positive-going parameters and negative-going parameters.

In some implementations, the test sequence comprises stepped-up stimulus intensity parameters. In some implementations, determining the first values for the one or more parameters of the model of INA of the neural pathway comprises determining, from (i) the stepped-up stimulus intensity parameters and (ii) the measured intensities of the neural responses evoked by the test sequence, first values for the positive-going parameters.

In some implementations, the test sequence comprises stepped-down stimulus intensity parameters. In some implementations, determining the first values for the one or more parameters of the model of INA of the neural pathway comprises determining, from (i) the stepped-down stimulus intensity parameters and (ii) the measured intensities of the neural responses evoked by the test sequence, first values for the negative-going parameters.

In some implementations, the method further comprises classifying the patient into one of a plurality of categories based on the first values for the parameters of the model of INA. In some implementations, the plurality of categories comprises responders to neural stimulation therapy, and non-responders to neural stimulation therapy.

In some implementations, the method further comprises predicting one or more characteristics of a patient's response to neural stimulation therapy based on the first values for the parameters of the model of INA.

In some implementations, the method further comprises repeating the delivering, capturing, measuring, and determining further values for the one or more parameters of the model of INA, and monitoring a progression of a condition of the patient based on the first values and the further values for the one or more of the parameters of the model of INA.

In some implementations, the method further comprises programming a neural stimulation system to deliver therapeutic neural stimuli to the neural pathway based on the first values for the one or more parameters.

Some aspects of the present disclosure relate to a neural stimulation system comprising an implantable device. The implantable device comprises a pulse generator configured to deliver neural stimuli to a neural pathway of a patient, the neural stimuli being configured to evoke neural responses from the neural pathway, and measurement circuitry configured to capture signal windows from signals sensed on the neural pathway by one or more measurement electrodes. The implantable device further comprises a controller. The controller is configured to control the pulse generator to deliver each neural stimulus, and control the measurement circuitry to capture a signal window from a signal sensed on the neural pathway subsequent to each neural stimulus. The neural stimulation system further comprises a test module comprising a processor. The test module is configured to cause the controller to control the pulse generator to deliver a test sequence of neural stimuli, each neural stimulus of the test sequence being delivered according to a respective stimulus intensity parameter, and cause the controller to control the measurement circuitry to capture a signal window from a signal sensed on the neural pathway subsequent to each neural stimulus of the test sequence. The test module is further configured to, for each delivered neural stimulus of the test sequence, measure an intensity of a neural response evoked by that neural stimulus from a signal window captured subsequent to the delivery of that neural stimulus. The test module is further configured to determine, from i) the respective stimulus intensity parameters of the test sequence and ii) the measured intensities of the neural responses evoked by the test sequence, first values for one or more parameters of a model of instantaneous neural accommodation (INA) of the neural pathway.

In some implementations, determining the first values for the one or more parameters comprises applying a regression model to the stimulus intensity parameters and the measured intensities of the neural responses. In some implementations, the controller is configured to deliver therapeutic neural stimuli to the neural pathway based on the first values for the one or more parameters.

In some implementations, the test module is further configured to classify the patient into one of a plurality of categories based on the first values of the parameters of the model of INA. In some implementations, the plurality of categories comprises responders to neural stimulation therapy, and non-responders to neural stimulation therapy. In some implementations, the test module is further configured to predict one or more characteristics of a patient's response to neural stimulation therapy based on the first values of the parameters of the model of INA.

Some aspects of the present disclosure relate to a neural stimulation system including a closed-loop neural stimulation device for controllably delivering neural stimuli to a neural pathway, the device having a controller configured to adjust, using a feedback controller having one or more loop parameters, a stimulus intensity of the delivered neural stimuli so as to maintain a measured intensity of neural responses evoked by the neural stimuli at or near a target response intensity. Neural stimulation system may also include a processor configured to: receive an INA characteristic vector having one or more parameters of a model of instantaneous neural accommodation of the neural pathway; and set the one or more loop parameters of the feedback controller of the closed-loop neural stimulation device based on the received INA characteristic vector.

Some aspects of the present disclosure relate to a method comprising receiving an INA characteristic vector having one or more parameters of a model of instantaneous neural accommodation of the neural pathway. The method may also include set the one or more loop parameters of the feedback controller of the closed-loop neural stimulation device based on the received INA characteristic vector.

Some aspects of the present disclosure relate to a closed-loop neural stimulation device including a controller configured to adjust, using a feedback controller having one or more loop parameters, a stimulus intensity of the delivered neural stimuli so as to maintain a measured intensity of neural responses evoked by the neural stimuli at or near a target response intensity. The closed-loop neural stimulation device may also include where the one or more loop parameters have been configured to achieve a stable low-pass response in the presence of instantaneous neural accommodation of the neural pathway.

Some aspects of the present disclosure relate to a method comprising delivering a test sequence of neural stimuli to the neural pathway, each neural stimulus being configured to evoke a neural response from the target neural fibres, each neural stimulus being delivered according to a stimulus intensity parameter. The method may also include capturing a signal window from each signal sensed from the target neural fibres subsequent to the respective neural stimuli. The method may furthermore include measuring intensities of neural responses evoked by the respective neural stimuli in the signal windows. The method may in addition include determining, from the stimulus intensity parameters and the respective measured intensities of the evoked neural responses, one or more key parameters of the response of the target neural fibres. The method may moreover include where the test sequence is configured such that the key parameters represent either: an accommodated response, or an un-accommodated response of the target neural fibres.

Some aspects of the present disclosure relate to a closed-loop neural stimulation device including a controller configured to adjust, using a feedback controller, a stimulus intensity of the delivered neural stimuli so as to maintain a property of the neural pathway at or near a target value so as to achieve a more stable sensation of stimulation as posture varies in the presence of instantaneous neural accommodation (INA) of the neural pathway.

The present technology has been developed primarily for use in or with neuromodulation of the spinal cord and will be described hereinafter mostly with reference to this application. However, it will be appreciated that the present technology is not limited to this particular field of use, and may be applied in other neuromodulation contexts, including but not limited to sacral nerve stimulation, pudendal nerve stimulation, deep brain stimulation, stimulation of other parts of the peripheral and central nervous system. It will further be appreciated that the present technology may be applied for treatment of conditions other than chronic pain, including but not limited to movement disorders, Crohn's disease, rheumatoid arthritis, diabetes, Reynaud's phenomenon, pelvic floor disorders, chronic inflammatory conditions, migraine, stroke, or depression.

### BRIEF DESCRIPTION OF THE DRAWINGS

Notwithstanding any other implementations which may fall within the scope of the present invention, one or more implementations of the present invention will now be described, by way of example only, with reference to the accompanying drawings, in which:

FIG. 1 schematically illustrates an implanted neural stimulator, according to one implementation of the present technology;

FIG. 2 is a block diagram of the stimulator of FIG. 1;

FIG. 3 is a schematic illustrating interaction of the implanted stimulator of FIG. 1 with a bundle of target nerve fibres;

FIG. 4 illustrates an idealised activation plot for one posture of a patient undergoing neural stimulation;

FIG. 5 is a schematic illustrating elements and inputs of a closed-loop neural stimulation (CLNS) system implementable by the electronics module of FIG. 2;

FIG. 6 illustrates the typical form of an electrically evoked compound action potential (ECAP) of a healthy subject;

FIG. 7 is a block diagram of a neural stimulation therapy system including the implanted stimulator of FIG. 1, according to an aspect of the present technology;

FIG. 8 is a graph showing a possible effect of instantaneous neural accommodation on collected stimulus/response data;

FIG. 9 is a schematic illustrating elements and inputs of a closed-loop neural stimulation (CLNS) system, according to an aspect of the present technology; and

FIG. 10a is a graph containing time traces illustrating increasing and decreasing time series models respectively of instantaneous neural accommodation, according to an aspect of the present technology;

FIG. 10b is a graph containing time traces illustrating closed-loop response intensity sequences resulting from increasing and decreasing steps in the target response intensity respectively, using the closed-loop neural stimulation (CLNS) system of FIG. 9, according to an aspect of the present technology; and

FIG. 11 is a flowchart illustrating a method of determining values for parameters of a model of instantaneous neural accommodation of target neural tissue, according to one aspect of the present technology.

### DETAILED DESCRIPTION OF THE PRESENT TECHNOLOGY

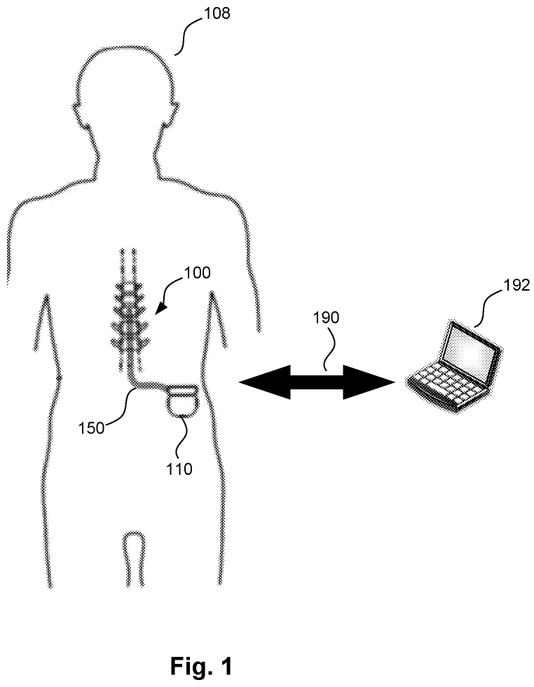

FIG. 1 schematically illustrates an implanted neural stimulator 100 in a patient 108, according to one implementation of the present technology. Stimulator 100 comprises an electronics module 110 housed within a conductive case, implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. The electronics module 110 is configured to electrically connect to an electrode assembly, typically comprising an electrode array 150 implanted within the epidural space and connected to the module 110 by a suitable lead. The electrode array 150 may comprise one or more electrodes such as electrode pads on a paddle lead, circular (e.g., ring) electrodes surrounding the body of a percutaneous lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

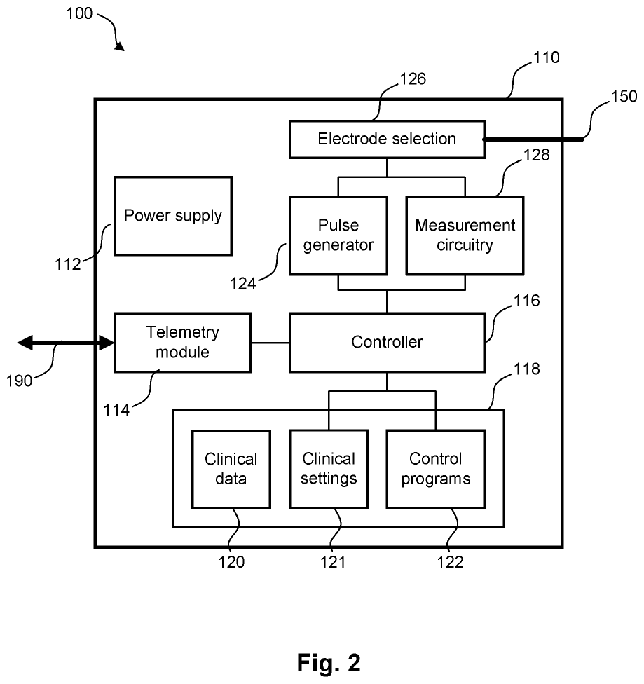

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive or inductive transfer, may be used by telemetry module 114 to transfer power or data to and from the electronics module 110 via communications channel 190. The controller 116 has an associated memory 118 storing one or more of clinical data 120, clinical settings 121, control programs 122, and the like. The controller 116 may comprise one or more processors, configured to, individually or in combination, execute machine-readable code, to cause the electronics module 110 to perform one or more methods described herein. The controller 116 is configured by control programs 122, sometimes referred to as firmware, to control a pulse generator 124 to generate stimuli, such as in the form of electrical pulses, in accordance with the clinical settings 121. Electrode selection module 126 switches the generated pulses to the selected electrode(s) of electrode array 150, for delivery of the pulses to the tissue surrounding the selected electrode(s). Measurement circuitry 128, which may comprise an amplifier or an analog-to-digital converter (ADC), is configured to process signals comprising neural responses sensed by measurement electrode(s) of the electrode array 150 as selected by electrode selection module 126.

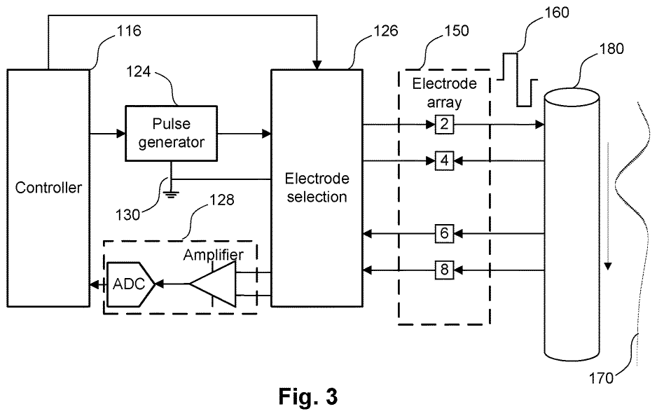

FIG. 3 is a schematic illustrating interaction of the implanted stimulator 100 with a bundle of target neural fibres 180 in the patient 108. In the implementation illustrated in FIG. 3 the target fibres 180 may be located in the spinal cord, however in alternative implementations the stimulator 100 may be positioned adjacent any target neural tissue including a peripheral nerve, visceral nerve, sacral nerve, parasympathetic nerve, or a brain structure. Electrode selection module 126 selects a stimulus electrode 2 of electrode array 150 through which to deliver a pulse from the pulse generator 124 to surrounding neural tissue including target fibres 180. A pulse may comprise one or more phases, e.g. a monophasic pulse comprises one phase, and a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative implementations may apply other forms of bipolar stimulation, or may use a greater number of stimulus or return electrodes. By contrast, in monopolar stimulation, current is returned through the conductive case of the stimulator 100, which may therefore be configured and function as an electrode though it is not physically part of the electrode array 150. The set of stimulus electrodes and return electrodes (collectively referred to as stimulation electrodes) is referred to as the stimulus electrode configuration. Electrode selection module 126 is illustrated as connecting to a ground 130 of the pulse generator 124 to enable stimulus current return via the return electrode 4. However, other connections for current return may be used in other implementations.

Delivery of an appropriate stimulus via stimulation electrodes 2 and 4 to the target fibres 180 evokes a neural response 170 comprising an evoked compound action potential (ECAP) which will propagate along the target fibres 180 as illustrated at a rate known as the conduction velocity. The ECAP may be evoked for therapeutic purposes, which in the case of a spinal cord stimulator for chronic pain may be associated with paresthesia at a desired location. To this end, the electrodes 2 and 4 are used to deliver stimuli periodically at any therapeutically suitable stimulus frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that may be experienced by the patient as paresthesia. When a stimulus electrode configuration is found which evokes paresthesia in a location and of a size which is congruent with the area of the patient's body affected by pain and of a quality that is comfortable for the patient, the clinician or the patient nominates that configuration for ongoing use. The therapy parameters may be loaded into the memory 118 of the electronics module 110 as the clinical settings 121.

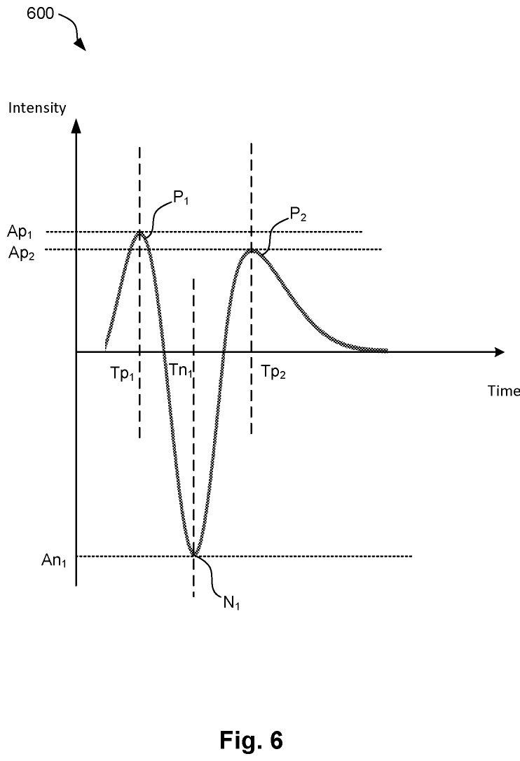

FIG. 6 illustrates the typical form of an ECAP 600 of a healthy subject, as sensed by a single measurement electrode referenced to the system ground 130 or referenced to an indifferent electrode. Such configurations are referred to as single-ended ECAP measurement. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap1 and occurs at time Tp1. The amplitude of the positive peak P2 is Ap2 and occurs at time Tp2. The amplitude of the negative peak N1 is An1 and occurs at time Tn1. The peak-to-peak amplitude is Ap1+An1. A recorded ECAP will typically have a maximum peak-to-peak amplitude in the range of microvolts and a duration of 2 to 3 ms. Characteristics of an evoked neural response (e.g., characteristics of an ECAP) may comprise one or more of, but not limited to: an amplitude of a first positive peak P1; an amplitude of a second positive peak P2; an amplitude of the negative peak N1; and a peak-to-peak or root-mean-square amplitude of the ECAP.

The stimulator 100 is further configured to measure the intensity of ECAPs 170 propagating along target fibres 180, whether such ECAPs are evoked by the stimulus from electrodes 2 and 4, or otherwise evoked. To this end, any electrodes of the electrode array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the target fibres 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (μV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response.

Stimulator 100 applies stimuli over a potentially long period such as days, weeks, or months and during this time may store characteristics of neural responses, clinical settings, target response intensity, and other operational parameters in memory 118. To effect suitable neural stimulation therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

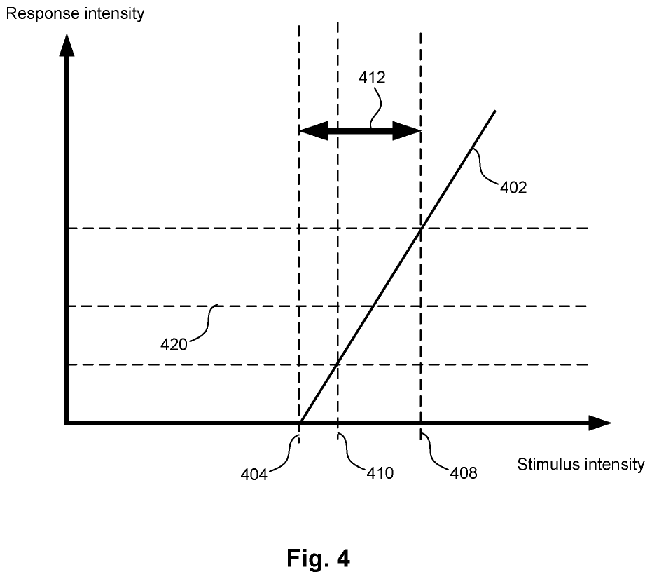

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4 illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled in piecewise linear form as:

$$d = \{ \begin{matrix}S ( s - T ) , & s \geq T \\ 0 , & s < T\end{matrix} \qquad ( 1 )$$

where s is the stimulus intensity, d is the ECAP amplitude, Tis the ECAP threshold and S is the slope of the activation plot (referred to herein as the patient sensitivity) above the ECAP threshold T. The sensitivity S and the ECAP threshold T are the key parameters of the activation plot 402.

FIG. 4 also illustrates a discomfort threshold 408, which is a stimulus intensity above which the patient 108 experiences uncomfortable or painful stimulation. FIG. 4 also illustrates a perception threshold 410. The perception threshold 410 is a value of stimulus intensity that corresponds to an ECAP amplitude that is barely perceptible by the patient. There are a number of factors which can influence the position of the perception threshold 410, including the posture of the patient. Perception threshold 410 may correspond to a stimulus intensity that is greater than the ECAP threshold 404, as illustrated in FIG. 4, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal-to-noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108, becoming steeper in more sensitive postures and shallower in less sensitive postures.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the feedback variable at or near a target response intensity. For example, the device may calculate an error between a target ECAP amplitude and a measured ECAP amplitude, and adjust the applied stimulus intensity to bring the measured ECAP amplitude closer to the target ECAP amplitude, such as by adding the scaled error to the current stimulus intensity. A neuromodulation device that operates by adjusting the applied stimulus intensity to maintain a feedback variable at or near a target value is said to be operating in closed-loop mode and will also be referred to as a closed-loop neural stimulation (CLNS) device. By adjusting the applied stimulus intensity to maintain the measured ECAP amplitude at or near an appropriate target response intensity, such as a target ECAP amplitude 420 illustrated in FIG. 4, a CLNS device will generally keep the stimulus intensity within the therapeutic range as patient posture varies.

A CLNS device comprises a pulse generator that takes a stimulus intensity value and converts it into neural stimuli comprising a sequence of electrical pulses according to a predefined stimulation pattern. The stimulation pattern is parametrised by multiple stimulus parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the pulse generator to implement the received stimulus intensity value. For example, all stimulus parameters may be held constant except stimulus amplitude which is determined in proportion to the received stimulus intensity value. Alternatively, all stimulus parameters may be held constant except pulse width which is determined in proportion to the received stimulus intensity value.

In an example CLNS system, the user sets a target response intensity, and the CLNS device performs proportional-integral-differential (PID) control. In some implementations, the differential and proportional contributions are disregarded and the CLNS device uses a first order integrating feedback loop. The pulse generator generates a stimulus in accordance with a stimulus intensity parameter, which evokes a neural response in the patient. The intensity of an evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at or near the target response intensity. If the target response intensity is properly chosen, the patient receives consistently comfortable and therapeutic stimulation through posture changes and other perturbations to the stimulus/response behaviour.

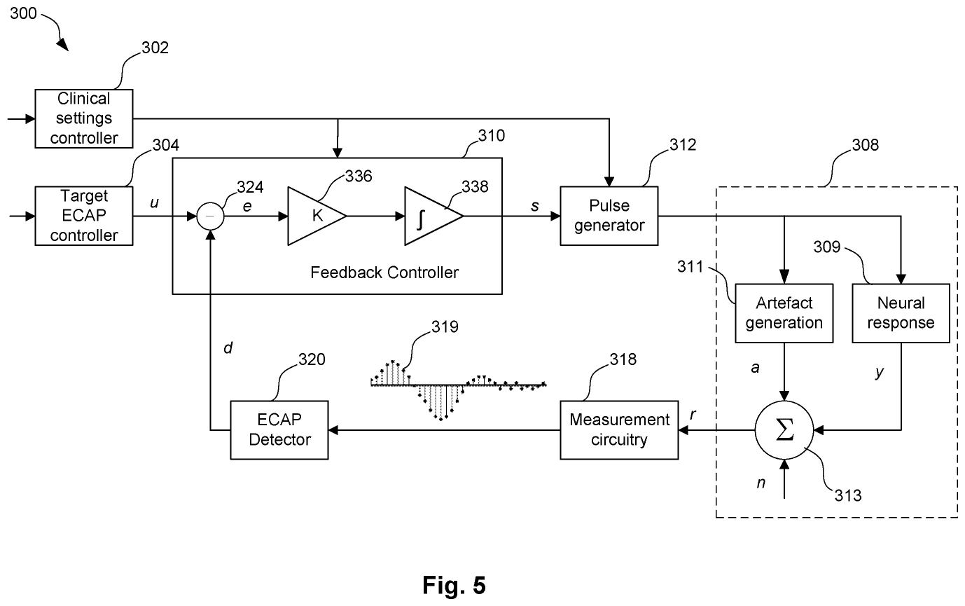

FIG. 5 is a schematic illustrating elements and inputs of a closed-loop neural stimulation (CLNS) system 300, implementable in part by the electronics module 110 of FIG. 2, according to one implementation of the present technology. The system 300 comprises a pulse generator 312 which converts a stimulus intensity parameter (for example a stimulus current amplitude) s, in concert with a set of predefined stimulus parameters, into neural stimuli comprising a sequence of electrical pulses delivered via the stimulation electrodes (not shown in FIG. 5). According to one implementation, the predefined stimulus parameters comprise the number and order of phases, the number of stimulus electrode poles, the pulse width, and the stimulus rate or frequency, and the pulse generator 312 determines the stimulus amplitude in proportion to the stimulus intensity parameter s.

The generated stimulus crosses from the electrodes to the neural tissue, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (potentially including evoked neural response, artefact, and measurement noise), and samples the amplified sensed signal to capture a “signal window” 319 comprising a predetermined number of samples of the amplified sensed signal. The ECAP detector 320 processes the signal window 319 and outputs a measured neural response intensity d. In one implementation, the neural response intensity comprises a peak-to-peak ECAP amplitude. The measured response intensity d (an example of a feedback variable) is input into the feedback controller 310. The feedback controller 310 comprises a comparator 324 that compares the measured response intensity d to a target ECAP amplitude u as set by the target ECAP controller 304 and provides an indication of the difference between the measured response intensity d and the target ECAP amplitude u. This difference is the error value, e.

The feedback controller 310 calculates an adjusted stimulus intensity parameter, s, with the aim of maintaining the measured response intensity d at or near the target ECAP amplitude u. Accordingly, the feedback controller 310 adjusts the stimulus intensity parameter s to minimise the error value, e. In one implementation, the feedback controller 310 utilises a first order integrating function, using a gain element 336 and an integrator 338, in order to provide suitable adjustment to the stimulus intensity parameter s. According to such an implementation, the current stimulus intensity parameter s may be determined by the feedback controller 310 as

s=∫Kedt (2)
where K is the gain of the gain element 336 (the controller gain). This relation may also be represented as

δs=Ke (3)
where δs is an adjustment to the current stimulus intensity parameter s.

A target ECAP amplitude u is input to the feedback controller 310 by or via the target ECAP controller 304. In one implementation, the target ECAP controller 304 provides an indication of a specific target ECAP amplitude. In another implementation, the target ECAP controller 304 provides an indication to increase or to decrease the present target ECAP amplitude. The target ECAP controller 304 may comprise an input into the CLNS system 300, via which the patient or clinician can input a target ECAP amplitude, or indication thereof. The target ECAP controller 304 may comprise memory in which the target ECAP amplitude is stored, and from which the target ECAP amplitude is provided to the feedback controller 310.

A clinical settings controller 302 provides clinical settings to the system 300, including the feedback controller 310 and the stimulus parameters for the pulse generator 312 that are not under the control of the feedback controller 310. In one example, the clinical settings controller 302 may be configured to adjust the controller gain K of the feedback controller 310 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 16 kHz). As the ECAP detector 320 is linear, only the stimulus clock affects the dynamics of the CLNS system 300. On the next stimulus clock cycle, the pulse generator 312 generates a stimulus in accordance with the adjusted stimulus intensity s. Accordingly, there is a delay of one stimulus clock cycle before the stimulus intensity delivered to the patient is updated in light of the error value e.

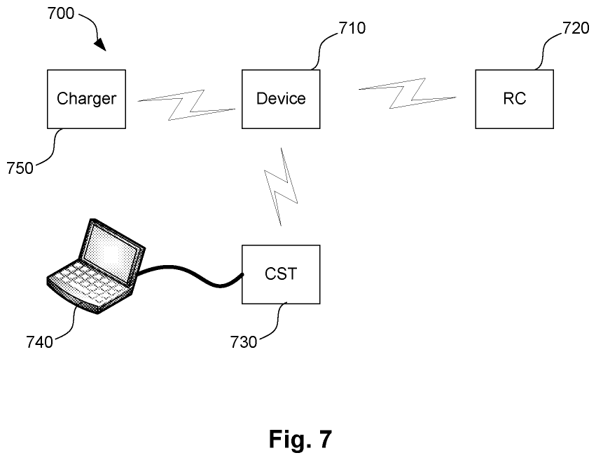

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

### A Measure of Neural Activation

Neural activation refers to the number of fibres recruited by a stimulus provided by a neural stimulator. The stimulus current flowing through the one or more stimulus electrodes to the one or more return electrodes generates a field at the neural tissue (e.g., the spinal cord). It will be appreciated that for a given stimulus intensity, as the stimulus electrode changes distance with respect to the cord, the field intensity at the neural tissue changes. The stimulus intensity at which the field generated by the stimulus begins to recruit fibres, for a specific posture of a patient, is the activation threshold (or ECAP threshold) for that posture of the patient.

The recruitment of fibres begins at approximately the same field intensity at the spinal cord regardless of the patient's posture. Therefore, the ECAP threshold for a patient's current posture provides a calibration point for estimating the neural activation resulting from stimuli at a given stimulus intensity. At stimulus intensities above the ECAP threshold, the neural activation of the field generated by the stimulus increases with the ratio by which the stimulus intensity exceeds the ECAP threshold. A measure D of neural activation resulting from stimuli at a stimulus intensity s may therefore be determined as the “dose ratio” of s to the ECAP threshold T:

$$D = \frac{s}{T} \qquad ( 4 )$$

It has been hypothesised that the dose ratio defined by equation (4) is a measure of dose that is normalised across all patients, i.e. delivery of a similar dose will have a similar analgesic effect on different patients, and on the same patient in different postures. It has further been demonstrated that dose ratio D is correlated with patient analgesic effect and that a dose ratio of approximately 1.4 is a threshold of therapeutic efficacy across many patients.

### Instantaneous Neural Accommodation

As mentioned above, it may be the case that neural fibres have some degree of memory even over time scales of tens of milliseconds. In other words, the piecewise linear model of Equation (1) for the relation between stimulus intensity s and response intensity d may not hold uniformly for stimulation frequencies in the tens of Hz range. Furthermore, no model not involving the history of recent stimuli as well as the current stimulus may adequately capture the relation between s and d.

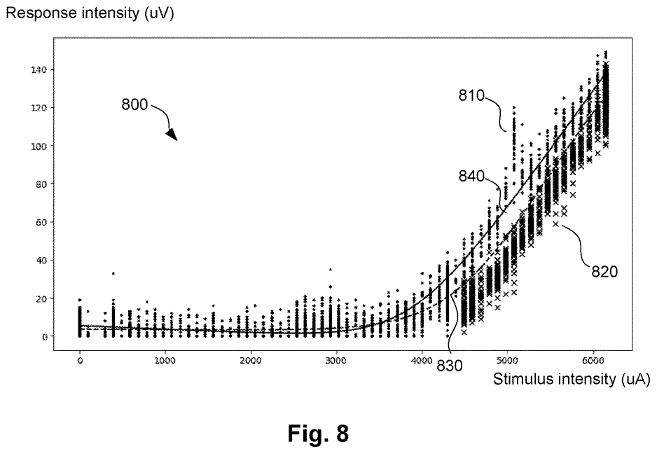

FIG. 8 contains a graph 800 showing evidence that this may be the case. The graph 800 contains a set 810 of (s, d) points (indicated by dots) captured from stimuli whose intensities s follow an “upward sweep” of stimulus intensity. The graph 800 also contains a set 820 of (s, d) points (indicated by crosses) captured from stimuli whose intensities s follow an “downward sweep” of stimulus intensity that immediately followed the upward sweep corresponding to the set 810. It may be seen that the response intensities of the set 820 are generally lower for the same stimulus intensity than the response intensities of the set 810. One explanation is that the target neural fibres, having been previously stimulated during the upward sweep, are collectively somewhat “accommodated” to the stimuli and therefore respond less intensely to stimuli of equal intensity during the downward sweep. This may be because at least some of the target fibres still have subnormal excitability when the stimuli corresponding to the set 820 are delivered and therefore may or may not activate in response to those stimuli. This accommodation is on the time scale of seconds, and possibly shorter, and is referred to herein as “instantaneous neural accommodation” (INA) to distinguish the phenomenon from other phenomena of longer time scales also sometimes referred to as “neural accommodation” (or “habituation” or “tolerance”).

The graph 800 also contains an activation plot 830 that has been fitted (according to a more sophisticated model than equation (1) that includes a curved transition portion between two linear portions) to the entire ensemble of (s, d) points (i.e. both the set 810 and the set 820). Also shown in the graph 800 is an activation plot 840 that has been fitted to the set 810 alone, during which the target fibres are presumed to be somewhat less accommodated than during the set 820. It may be seen that the activation plots and 830 and 840 are different and will therefore have different key parameters such as sensitivity or threshold. Thus, the phenomenon of instantaneous neural accommodation has the potential to affect the estimation of key parameters of the target fibres' response to stimulation, if not taken carefully into account.

On the assumption that instantaneous neural accommodation does exist, the methods and systems according to aspects of the present technology are intended to quantify the phenomenon and use the resulting parameter values as biomarkers to screen for, monitor, program, and control closed-loop neural stimulation therapy.

One model for a relation between a discrete sequence of stimuli of intensity s[n] and a sequence of response intensities d[n] of a set of target fibres subject to instantaneous neural accommodation is a linear time invariant time series model known as autoregressive moving average (ARMA):

d[n]=Σ_{k≥0}a[k]s[n−k]+Σ_{k>0}b[k]d[n−k] (5)

The relation between s[n] and d[n] in Equation (5) may be represented in the Z-transform domain as a transfer function H(z) comprising a ratio of polynomials:

$$\frac{D ( z )}{S ( z )} = H ( z ) = \frac{A ( z )}{B ( z )} \qquad ( 6 )$$

The parameters of the ARMA model of INA are the coefficients a[k] of the numerator polynomial A(z) (representing the moving average) and the coefficients b[k] of the denominator polynomial B(z) (representing the autoregression). The piecewise linear relation in equation (1) is a special (memoryless) case of equation (5) in which (for values of s above the ECAP threshold T) a[0] is equal to the patient sensitivity S, b[0] is equal to one, and all other coefficients are zero.

The ARMA model parameters may be fitted to a particular stimulus electrode configuration (SEC) and measurement electrode configuration (MEC) at a given stimulus frequency by delivering a test sequence of stimuli of varying intensities s[n], such as according to a step, via the SEC at the stimulus frequency and measuring the resulting response intensity sequence d[n] at the MEC. In one example, the sequence s[n] (after subtracting the ECAP threshold T) may be deconvolved from the sequence d[n] to obtain an impulse response sequence h[n]. The impulse response sequence h[n] may be transformed to the Z-domain as the transfer function H(z) which in turn may be approximated as a ratio of polynomials A(z)/B(z), each of which may be limited to a predetermined number of coefficients a[k] and b[k] respectively. The coefficients a[k] and b[k] form a “characteristic vector” {a[k], b[k]} of parameters of the ARMA model of INA. The test sequence may be predetermined or may be adaptive to the response intensity sequence d[n].

The ARMA model parameters may be determined as a result of applying a test sequence comprising a step of the stimulus intensity parameters of the neural stimuli of the test sequence. In some implementations, a step comprises an increase of stimulus intensity (such as a step up). In some implementations, a step comprises a decrease of stimulus intensity (such as a step down).

Instantaneous neural accommodation may be an asymmetrical phenomenon. That is, the parameters of the time series model of INA may be different depending on whether the stimulus intensity is increasing or decreasing. Therefore, in some implementations, the above ARMA model fitting is performed twice, once using an increase of stimulus intensity (such as a step up) and once using a decrease of stimulus intensity (such as a step down). The result in such implementations is a bifurcated ARMA model of INA comprising a characteristic vector comprising two sets of parameters: the “positive-going” parameters {a⁺[k], b⁺[k]} for increasing stimulus intensity and the “negative-going” parameters {a⁻[k], b⁻[k]} for decreasing stimulus intensity.

In yet other implementations, the parameters of the time series model of INA may be different depending on the start and end intensities of the change in stimulus intensity. In some such implementations, the above ARMA model fitting may be performed once for each pair of start and end intensities within a range of stimulus intensities. The result of such implementations is a “matrix” ARMA model of INA comprising a matrix, each cell of which corresponds to a (start intensity, end intensity) pair. Each cell of the matrix contains a characteristic vector {a[k], b[k]} corresponding to the ARMA model parameters at the corresponding (start intensity, end intensity).

Other time series models besides ARMA are contemplated. In another implementation, the time series model is an initial step up followed by a decaying exponential to approach a lower final value (for an increase of stimulus intensity) or an initial step down followed by a constant minus a decaying exponential to approach a higher final value (for a decrease of stimulus intensity).

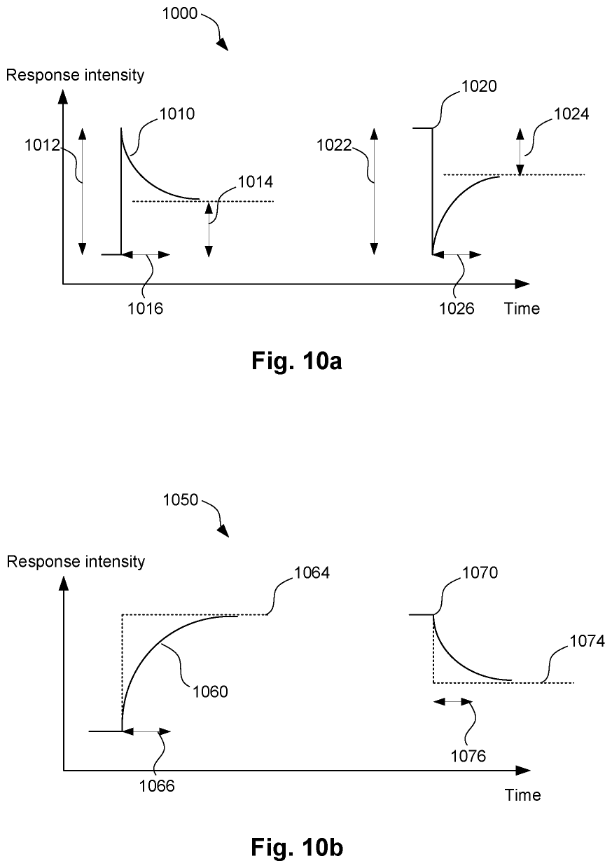

FIG. 10a is a graph 1000 containing time traces 1010 and 1020 illustrating increasing and decreasing time series models, respectively. The parameters of the increasing time series model 1010 are the size 1012 of the initial step and the size 1014 of the final step, relative to the size of the stimulus intensity step (which may be normalised to the ECAP threshold) and the time constant 1016 of the exponential decay. The decreasing time series model 1020 has three analogous parameters, illustrated as initial step size 1022, final step size 1024, and time constant 1026. These parameters may be estimated for a particular SEC and MEC at a given stimulus frequency by delivering a test sequence of stimuli of varying intensities s[n], such as a step up of a predetermined size (which may be normalised to the ECAP threshold), via the SEC at the stimulus frequency and measuring the resulting response intensity sequence d[n] from the signal windows sensed at the MEC. Stimulus intensity parameters that comprise increasing stimulation intensities s[n], such as a step up of a predetermined size, may be referred to as stepped-up stimulus intensity parameters. In some implementations, the test sequence comprises stepped-up stimulus intensity parameters, and determining the values of the INA model parameters comprises determining, from the stepped-up stimulus intensity parameters and the respective measured intensities of the evoked neural responses, values of the positive-going INA model parameters.

The increasing time series model represented by the trace 1010 may be fitted, such as by least squares fitting, to the response intensity sequence d[n] to determine values for the positive-going INA model parameters 1012, 1014, and 1016. The same procedure may be carried out for a step down in stimulus intensities of a predetermined size (which may be normalised to the ECAP threshold) to obtain the negative-going INA model parameters 1022, 1024, and 1026 of the decreasing time series model. Stimulus intensity parameters that comprise decreasing stimulation intensities s[n], such as a step down of a predetermined size, may be referred to as stepped-down stimulus intensity parameters. In some implementations, the test sequence comprises stepped-down stimulus intensity parameters, and determining the values of the INA model parameters comprises determining, from the stepped-down stimulus intensity parameters and the respective measured intensities of the evoked neural responses, values of the negative-going INA model parameters.

The characteristic vector in a bifurcated INA model comprises the positive-going and negative-going INA model parameters.

In some implementations, the parameters of the time series model of INA may be converted to parameters of a collective Hodgkin-Huxley model of the target neural fibres. The characteristic vector of the INA model of the target neural fibres then comprises the Hodgkin-Huxley model parameters.

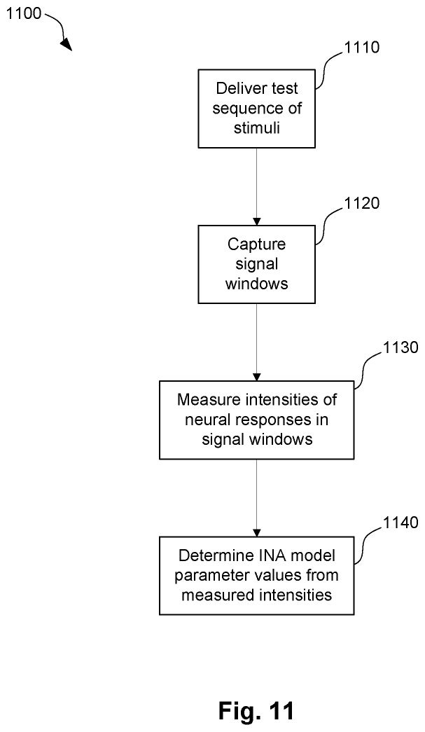

FIG. 11 is a flowchart illustrating a method 1100 of determining values for parameters of a model of INA of the target neural fibres, according to one aspect of the present technology. The method 1100 may be performed by a test module.

In some implementations, the test module comprises the controller 116 of the electronics module 110 of the stimulator 100, wherein the test module is configured by instructions stored in the control programs 122 of the memory 118.

In another implementation, the test module comprises a processor of the external device 192 in communication with the controller 116 of the electronics module 110. The communication may comprise transcutaneous communication. The test module is configured to cause the controller 116 to perform steps in accordance with methods described herein.

In another implementation, the test module comprises the controller 116 in combination with a processor of the external device 192. In such an implementation, the controller 116 and the processor of the external device 192 are configured to, in combination, perform the method 1100. In such an implementation, the processor of the external device 192 may be configured to transmit, to the controller 116, via the communication channel 190, parameters that characterise the test sequence. The controller 116 may be configured to transmit test data to the processor of the external device 192, via communication channel 190. The test data may comprise one or more of: an indication of the measured intensities; and an indication of the signal windows captured by the measurement circuitry. The processor of the external device 192 may be configured to perform step 1140 to determine, from the stimulus intensity parameters and the measured intensities of the evoked neural responses, values for the one or more parameters of the model of INA of the neural pathway.

In step 1110, the test module causes the controller 116 to control the pulse generator to deliver a test sequence of neural stimuli to the target neural fibres. Each neural stimulus is delivered according to a respective stimulus intensity parameter. In step 1120, the test module causes the controller 116 to control the measurement circuitry to capture a respective signal window subsequent to each delivered neural stimulus. In step 1130, the test module measures intensities of neural responses evoked by the stimuli in the respective signal windows, and associates each measured response intensity with the stimulus intensity parameter value of the corresponding stimulus. In some implementations, in step 1130, the test module causes the controller 116 to measure intensities of neural responses evoked by the stimuli in the respective signal windows, and the controller 116 communicates data indicative of the measured intensities to the test module.

In step 1140, the test module determines values for one or more parameters of a model of INA of the target neural fibres from the measured response intensities and the corresponding stimulus intensity parameter values. In some embodiments, the test module applies a regression model to the measured response intensities and the corresponding stimulus intensity parameter values to determine values for one or more parameters of the model of INA of the target neural fibres. The regression model may comprise one or more of: a least squares regression model; a linear time invariant time model; an autoregressive model; and an autoregressive moving average model.

In some implementations, one or more of the parameters in the INA characteristic vector may be used as part of a differential diagnosis of the patient's condition, such as spinal cord health/injury, multiple sclerosis, amyotrophic lateral sclerosis, or other syndromes involving sodium or potassium channel disruption, disease, or mutation.

In some implementations, the INA characteristic vector may be used at the very early stage of patient programming to “phenotype” or “screen” the patient. That is, one or more of the parameters in the INA characteristic vector may be used to classify the patient into one of a plurality of categories for the purposes of predicting responder status to neural stimulation therapy, predicting whether the patient may be successfully programmable using automated rather than manual programming methods, or selecting a programming workflow from a plurality of possible workflows. The plurality of categories may comprise: responders to neural stimulation therapy; and non-responders to neural stimulation therapy.

In some implementations, one or more of the parameters in the INA characteristic vector may be used to predict characteristics of the patient's response to neural stimulation therapy. A characteristic of the patient's response may comprise one or more of: a as wash-in time (the time taken for neural stimulation therapy to take effect); and a wash-out time (the time taken for the effect of the neural stimulation therapy to decay after the neural stimulation therapy is switched off).

The above-described methods may be carried out repeatedly to obtain the INA characteristic vector as the patient undergoes neural stimulation therapy. The values of one or more of the parameters in the characteristic vector may be used across time as a biomarker to monitor the progression of the patient's condition or the therapeutic efficacy of the neural stimulation therapy.

As mentioned above, the key parameters of an activation plot are patient sensitivity (slope) and ECAP threshold. However, as illustrated in the graph 800 in FIG. 8, the fitting of the AP may be affected by instantaneous neural accommodation during the capturing of the data points to be fitted to an AP model. In particular, key parameters of the AP such as ECAP threshold and patient sensitivity may be affected by INA. This in turn may affect the ability of a CLNS device to maintain a consistent predetermined amount of neural activation, since dose ratio is determined in part by ECAP threshold (see equation (4)).

Implementations according to one aspect of the present technology are configured to capture data points for fitting an AP in a way that mitigates the effect of INA. In one implementation, which may be used without prior knowledge of the INA characteristic vector, the pulse generator 124 is instructed by the CPA to deliver a sequence of isolated individual stimuli of varying stimulus intensity parameter values s[n], separated from each other by a time interval that is longer than the time period required for excitability of the target fibres to return to its baseline state. For A-beta fibres this time period may be of the order of up to 100 to 150 milliseconds. The stimulus intensity parameter values s[n] may vary across the expected therapeutic range, such as according to an upward ramp or stepped ramp. The ECAP detector measures the intensity d[n] of the response evoked by each stimulus of intensity s[n]. An AP may then be fitted to the resulting set {(s[n], d[n])} of data points, which may be substantially unaffected by INA. The resulting key parameters may therefore represent the un-accommodated AP of the target fibres.

In another implementation according to this aspect, the pulse generator 124 is instructed by the CPA to deliver a sequence of individual stimuli at the chosen stimulus frequency, with a constant stimulus intensity s₁ at the high end of the expected therapeutic range. Preferably, the sequence lasts for an interval that is longer than the expected time constant of INA, so that the target fibres can be assumed to be accommodated by the end of the interval. The intensity d₁ of the response evoked by one or more stimuli at the end of this sequence is measured (if more than one value is measured, d₁ may be set to a representative value such as an average from the plurality of response intensities). The stimulus intensity s₁ is then decremented by a small amount to s₂, and the process repeated until the stimulus intensity is well below the expected ECAP threshold for the target fibres. An AP may then be fitted to the resulting set {(sᵢ, dᵢ)} of data points. The resulting key parameters represent an accommodated AP of the target fibres.

In another implementation, the set {(s[n], d[n])} of data points may be captured from stimuli delivered according to an upward ramp of stimulus intensity s[n] followed by a downward ramp at the chosen stimulus frequency. The points captured on the upward ramp are distinguishable from those captured on the downward ramp, for example by a time stamp or a binary-valued tag. FIG. 8 illustrates such a set of captured data points {(s[n], d[n])}, with upward-ramped and downward-ramped data points respectively marked with dots and crosses. The points captured on the downward ramp (such as the set 820 in the graph 800) may be discarded and the AP fitted only to the points captured on the upward ramp (such as the set 810 in the graph 800). Alternatively, only the first data point captured at each stimulus intensity on the upward ramp may be retained for AP fitting. The resulting key parameters may represent the un-accommodated AP of the target fibres. Alternatively, only the first data point captured at each stimulus intensity on the downward ramp may be retained for AP fitting. The resulting key parameters may represent the accommodated AP of the target fibres.

Once the INA characteristic vector or the key parameters of the AP of the target fibres, whether un-accommodated, accommodated, or both, have been obtained, a neural stimulation system such as the CLNS system 300 may be programmed to deliver therapeutic stimuli based on the INA characteristic vector or the key parameters of the AP. In some implementations, the programming selects parameters of the feedback loop (loop parameters) of a CLNS system such as the CLNS system 300 based on the INA characteristic vector or the key parameters of the AP. For example, the controller gain K of the feedback controller 310 illustrated in FIG. 5 may be chosen based on the measured patient sensitivity S. Implementations of programming methods according to this principle are described in International Patent Publication no. WO2025175339 by the present applicant, the entire content of which is herein incorporated by reference. In some such implementations, the accommodated patient sensitivity may be used as the value of S as the accommodated state is more likely to be reflective of the state of the target fibres during therapy. In other such implementations, suitable for bifurcated INA models, values for two controller gains are chosen: a positive-going controller gain K⁺, based on the positive-going portion of the bifurcated INA model, to be used when the target response intensity exceeds the measured response intensity; and a negative-going controller gain K⁻, based on the negative-going portion of the bifurcated INA model, to be used when the measured response intensity exceeds the target response intensity.

Another implementation of programming CLNS therapy based on the INA characteristic vector comprises determining a schedule of therapy utilisation (e.g., on/off timing or duty cycle of therapy) depending on the wash-in and wash-out times predicted from the INA characteristic vector, as described above.

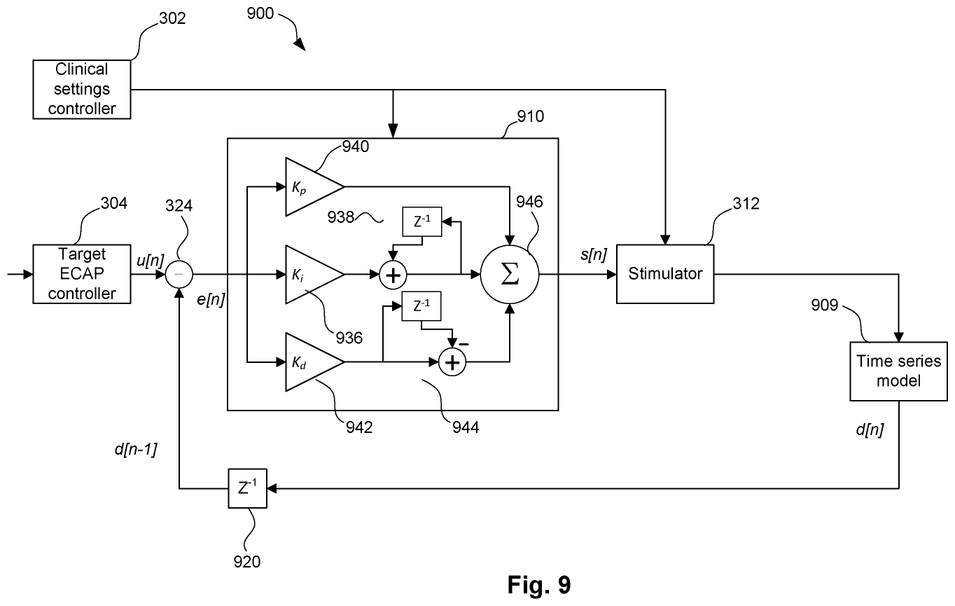

Other implementations of programming CLNS therapy based on the INA characteristic vector are suitable for a more elaborate form of feedback controller than the (integral) feedback controller 310 of FIG. 5. FIG. 9 is a schematic illustrating elements and inputs of an alternative CLNS system 900, according to an aspect of the present technology. The CLNS system 900 is similar to the CLNS system 300 of FIG. 5, with like labels indicating like elements, though the CLNS system 900 is illustrated as a discrete-time system. The box 309 has been replaced by a box 909 that incorporates the box 309, the measurement circuitry 318, and the ECAP detector 320, and therefore produces an evoked response intensity sequence d[n] in response to delivered stimuli represented by the stimulus intensity sequence s[n]. (Artefact and noise are ignored in FIG. 9.) The box 909 comprises a time series model that models the response sequence d[n] of target fibres subject to INA as evoked by the stimulus sequence of intensity s[n]. The delay element 920 delays the evoked response intensity sequence d[n] by one time interval before the comparator 324. The target ECAP controller 304 generates a sequence u[n] of target response intensities for the comparator 324 to compare with the delayed evoked response intensity sequence d[n−1].

The feedback controller 310 has been replaced by a discrete-time proportional-integral-differential (PID) controller 910 in the CLNS system 900. The integral portion of the PID controller 910 is implemented by an integral gain element 936 of gain Kᵢ followed by a first order discrete integrator 938. The proportional portion of the PID controller 910 is implemented by a proportional gain element 940 of gain Kₚ. The differential portion of the PID controller 910 is implemented by a differential gain element 942 of gain K_{d} followed by a first order discrete differentiator 944. The outputs of the three portions are summed by the summing element 946 to produce the stimulus intensity sequence s[n]. In other implementations, higher order integral or differential elements may be used in place of the first order elements 938 (discrete integrator) and 944 (discrete differentiator) respectively. The PID controller 910 has a controller gain transfer function G[z] with one pole at z=1 and two zeroes whose locations depend on the three gains Kₚ, Kᵢ, and K_{d}.

Programming the PID controller 910 is analogous to programming the integral feedback controller 310, except that there are three gains to be chosen (proportional gain Kₚ, integral gain Kᵢ, and differential gain K_{d}) rather than just the integral controller gain K in the integral feedback controller 310. The above-mentioned International Patent Publication no. WO2025175339 describes how to select a value for an integral controller gain K in order to achieve predetermined dynamic performance characteristics of the feedback loop depending on the patient sensitivity S (assuming a generally linear, memoryless time series model for the target fibres) subject to certain constraints. In general, the aim of the selection is to create a loop whose transfer function from target response intensity u[n] to measured response intensity d[n] has a single pole on the positive half of the real axis within the unit circle on the Z-plane, thereby achieving a stable, low-pass response to changes in the target response intensity u[n], e.g. changes resulting from operation of the RC 720, or changes in posture. Methods according to some implementations of one aspect of the present technology select the three gains Kₚ, Kᵢ, and K_{d} based on the INA time series model 909 in analogous fashion, to achieve the same aim of a stable, low-pass response. In some such implementations, suitable for an AR time series model of INA (represented by a transfer function H(z) as described above in which the MA or numerator polynomial A(z) is a scalar), the three gains Kₚ, Kᵢ, and K_{d} are chosen such that the zeroes of the controller transfer function G(z) substantially cancel the pole(s) of the AR model, i.e. the zeros of the denominator polynomial B(z) of the transfer function H(z). Such implementations may result in a closed-loop transfer function from target response intensity to evoked response intensity that achieves a stable, low-pass response.

FIG. 10b contains a graph 1050 containing time traces 1060 and 1070 illustrating closed-loop response intensity sequences d[n] resulting from increasing and decreasing steps 1064 and 1074 in the target response intensity u[n], respectively. To achieve the time traces 1060 and 1070, the PID controller transfer function G(z) has been programmed according to the above-described methods to achieve a stable, low-pass response. It may be seen that the trace 1060 exponentially rises to a final value of the increasing step 1064 with a time constant 1066. Likewise, the trace 1070 exponentially decays to a final value of the decreasing step 1074 with a time constant 1076. The traces 1060 and 1070 may be compared with the “open-loop” response intensity sequences 1010 and 1020 of FIG. 10a.

In some implementations, suitable for bifurcated INA models, the PID controller 910 is also bifurcated, comprising a positive-going portion (with gains K⁺ₚ, K⁺ᵢ, and K⁺_{d}) that is used when the target response intensity u[n] exceeds the measured response intensity d[n], and a negative-going portion (with gains K⁻ₚ, K⁻ᵢ, and K⁻_{d}) that is used when the measured response intensity d[n] exceeds the target response intensity u[n]. In such implementations, the positive-going controller gains K⁺ₚ, K⁺ᵢ, and K⁺_{d} are selected according to the positive-going parameters of the INA characteristic vector, and the negative-going controller gains K⁺ₚ, K⁺ᵢ, and K⁺_{d} are separately selected according to the negative-going parameters in the INA characteristic vector, both using the above-described methods.

Once therapy using the programmed CLNS system 900 has begun, the INA characteristic vector may be repeatedly measured using test stimuli, as described above. The above-described programming methods may be repeated to adjust the gains of the PID controller 910 to maintain the desired closed-loop transfer function based on the updated INA characteristic vector.

Alternatively, a system estimator such as a Kalman filter may be employed to simultaneously estimate the INA characteristic vector from the sequences of stimulus intensities s[n], target response intensities u[n], and response intensities d[n], and adjust the PID controller 910 accordingly to maintain the desired closed-loop transfer function.

Alternative implementations according to one aspect of the present technology are suitable for the CLNS system 300. However, such alternative implementations modify the above-described operation of the CLNS system 300 so that, in effect, it operates to achieve a target value that is not a response intensity of a bundle of target neural fibres, as the unmodified CLNS system 300 and the CLNS system 900 do, but a different property of the target neural fibres, such as volume of target fibres activated (VTA). Such a property in the presence of instantaneous neural accommodation is not necessarily reflected by an evoked response intensity, but may, if maintained at a target value by the implementations described below, result in effective therapy. Such implementations may provide a more stable sensation of stimulation to the patient (and hence a lower sensation of paresthesia) as posture varies in the presence of INA.

In one such implementation, a supervisory process such as the clinical settings controller 302 adjusts the stimulus intensities s[n] provided by the feedback controller 310 to adjusted stimulus intensities s′[n] before the pulse generator 312, to compensate for the effects of INA. In one example, after a significant upward adjustment to s[n] provided by the feedback controller 310 as a result of a change from a more sensitive to a less sensitive posture, the clinical settings controller 302 adjusts s[n] downward to prevent the further rise in s[n] that would otherwise result from the expected decrease in the response intensity d[n] after an increase in s[n] due to INA, as illustrated in the trace 1010. The amount of this downward adjustment is dependent on the INA characteristic vector, or (in a bifurcated INA model) the positive-going portion of the INA characteristic vector. Likewise, after a significant downward adjustment to s[n] provided by the feedback controller 310 as a result of a change from a less sensitive to a more sensitive posture, the clinical settings controller 302 adjusts s[n] upward to prevent the further fall in s[n] that would otherwise result from the expected increase in the response intensity d[n] after a decrease in s[n] due to INA, as illustrated in the trace 1020. The amount of this upward adjustment is dependent on the INA characteristic vector, or (in a bifurcated INA model) the negative-going portion of the INA characteristic vector.

In another such implementation, the target ECAP controller 302 adjusts the patient-controlled target sequence u[n] to an adjusted target sequence u′[n] before the comparator 324. In one example, after a change from a more sensitive to a less sensitive posture, the target ECAP controller 304 adjusts u[n] downward to prevent the further rise in s[n] that would otherwise result from the expected decrease in the response intensity d[n] after a step increase in s[n] due to INA, as illustrated in the trace 1010. The amount of this downward adjustment is dependent on the INA characteristic vector, or (in a bifurcated INA model) the positive-going portion of the INA characteristic vector. Likewise, after a change from a less sensitive to a more sensitive posture, the target ECAP controller 302 adjusts u[n] upward to prevent the further fall in s[n] that would otherwise result from the expected increase in the response intensity d[n] after a step decrease in s[n] due to INA, as illustrated in the trace 1020. The amount of this upward adjustment is dependent on the INA characteristic vector, or (in a bifurcated INA model) the negative-going portion of the INA characteristic vector.

### Interpretation

The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be implemented as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored or executed in a distributed fashion. The present technology is not limited to any particular programming language or operating system.

### Wireless

In the context of the present disclosure, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. In the context of the present disclosure, the term “wired” and its derivatives may be used to describe circuits, devices, systems, methods, techniques, communications channels, etc., that may communicate data through the use of modulated signals propagating through a conductive medium. The term does not imply that the associated devices are coupled by electrically conductive wires.

Wireless communication standards that can be accommodated include IEEE 802.11 wireless LANs and links, Bluetooth, and wireless Ethernet. The technology disclosed herein may be implemented using devices conforming to other network standards and for other applications, including, for example other WLAN standards and other wireless standards such as MICS.

### Processes

Unless specifically stated otherwise, as apparent from the following discussions, it is appreciated that throughout the specification discussions utilizing terms such as “processing”, “computing”, “comparing”, “estimating”, “calculating”, “determining”, “analysing” or the like, refer to the action or processes of a computer or computing system, or similar electronic computing device, that manipulate or transform data represented as physical, such as electronic, quantities into other data similarly represented as physical quantities, or to otherwise execute a predefined procedure suitable to effect the described actions.

### Processor

In a similar manner, the term “processor” may refer to any device or portion of a device that processes electronic data, e.g., from registers or memory, to transform that electronic data into other electronic data that, e.g., may be stored in registers or memory. A “computer” or a “computing device” or a “computing machine” or a “computing platform” may include one or more processors.

The methods described herein are, in one embodiment, performable by one or more processors, individually or in combination, that accept computer-readable (also called machine-readable) code containing a set of instructions that when executed by one or more of the processors cause the one or more processors to carry out at least one of the methods described herein. Any processor capable of executing a set of instructions (sequential or otherwise) that specify actions to be taken are included within the meaning of the term “processor”. Thus, one example is a typical processing system that includes one or more processors. The processing system further may include a memory subsystem including main RAM or a static RAM, or ROM.

### Networked or Multiple Processors

In alternative embodiments, the one or more processors operate as respective standalone device(s) or may be connected, e.g., networked to other processor(s), in a networked deployment. The one or more processors may operate in the capacity of a server or a client machine in server-client network environment, or as a peer machine in a peer-to-peer or distributed network environment. The one or more processors may form a web appliance, a network router, switch or bridge, or any machine capable of executing a set of instructions (sequential or otherwise) that specify actions to be taken by that machine.

Note that while some diagram(s) only show(s) a single processor and a single memory that carries the computer-readable code, those in the art will understand that many of the components described above are included, but not explicitly shown or described in order not to obscure the inventive aspect. For example, while only a single machine may be illustrated, the term “machine” shall also be taken to include any collection of machines that individually or jointly execute a set (or multiple sets) of instructions to perform any one or more of the methods discussed herein.

### Additional Implementations

Thus, one implementation of each of the methods described herein is in the form of a computer-readable medium carrying a set of instructions, e.g., a computer program that are for execution on one or more processors. Thus, as will be appreciated by those skilled in the art, aspects of the present technology may be implemented as a method, an apparatus such as a special purpose apparatus, an apparatus such as a data processing system, or a computer-readable medium. The computer-readable medium carries computer-readable code including a set of instructions that when executed on one or more processors cause the processor or processors, individually or in combination, to implement a method. Accordingly, aspects of the present technology may take the form of a method, an entirely hardware implementation, an entirely software implementation or an implementation combining software and hardware aspects. Furthermore, the present technology may take the form of a carrier medium (e.g., a computer program product) carrying computer-readable program code embodied in the medium.

### Carrier Medium

The software may further be transmitted or received over a network via a network interface device. While the carrier medium is shown in an example embodiment to be a single medium, the term “carrier medium” should be taken to include a single medium or multiple media (e.g., a centralized or distributed database, or associated caches and servers) that store the one or more sets of instructions. A carrier medium may take many forms, including but not limited to, non-volatile media, volatile media, and transmission media.

### Means for Carrying Out a Method or Function

Furthermore, some of the implementations are described herein as a method or combination of elements of a method that can be implemented by one or more processors of a processor device, computer system, or by other means of carrying out the function. Thus, one or more processors with the necessary instructions for carrying out such a method or element of a method, individually or in combination, forms a means for carrying out the method or element of a method. Furthermore, an element described herein of an apparatus is an example of a means for carrying out the function performed by the element.

Those of skill would further appreciate that the various illustrative logical blocks, modules, and algorithm steps described in connection with the implementations disclosed herein may be implemented as electronic hardware, computer software running on a special purpose machine that is programmed to carry out the operations described in the present disclosure, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the exemplary implementations.

### Implementations

Reference throughout the present disclosure to “one implementation” or “an implementation” means that a particular feature, structure or characteristic described in connection with the implementation is included in at least one implementation of the present technology. Thus, appearances of the phrases “in one implementation” or “in an implementation” in various places throughout the present disclosure are not necessarily all referring to the same implementation, but may refer to different implementations. Furthermore, the particular features, structures or characteristics may be combined in any suitable manner, as would be apparent to one of ordinary skill in the art from this disclosure, in one or more implementations.

Similarly, it should be appreciated that in the above description of example implementations of the present technology, various features are sometimes grouped together in a single implementation, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects may lie in less than all features of a single foregoing disclosed implementation. Thus, the claims following the Detailed Description of the Present Technology are hereby expressly incorporated into this Detailed Description of the Present Technology, with each claim standing on its own as a separate implementation of the present technology.

Furthermore, while some implementations described herein include some, but not other features included in other implementations, combinations of features of different implementations are meant to be within the scope of the present technology, and form different implementations of the present technology, as would be understood by those in the art. For example, in the following claims, any of the claimed implementations can generally be used in any combination.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude or position to indicate that the value or position described is within a reasonable expected range of values or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that each value between two particular values is also disclosed. For example, if 10 and 15 are disclosed, then 11, 12, 13, and 14 are also disclosed.

### Different Instances of Objects

As used herein, unless otherwise specified the use of the ordinal adjectives “first”, “second”, “third”, etc., to describe a common object, merely indicates that different instances of like objects are being referred to, and is not intended to imply that the objects so described must be in a given sequence, either temporally, spatially, in ranking, or in any other manner.

### Specific Details

In the description provided herein, numerous specific details are set forth. However, it is understood that implementations of the present technology may be practiced without these specific details. In other instances, well-known methods, structures and techniques have not been shown in detail in order not to obscure an understanding of the present technology.

### TERMINOLOGY

Throughout the present disclosure, the terms “a” and “an” mean “one or more”, unless expressly specified otherwise.

Throughout the present disclosure, the word “comprise”, or variations such as “comprises” or “comprising”, will be understood to imply the inclusion of a stated element, integer, or step, or group of elements, integers, or steps, but not the exclusion of any other element, integer, or step, or group of elements, integers, or steps.

Throughout the present disclosure, a statement that an element may be “at least one of” or “one or more of” a list of options is to be understood to mean that the element may be any one of the listed options, or may be any combination of two or more of the listed options.

Throughout the present disclosure, the word “or” is to be read inclusively rather than exclusively, except where otherwise indicated.

Neither the title nor any abstract of the present disclosure should be taken as limiting in any way the scope of the claimed invention.

Where the preamble of a claim recites a purpose, benefit or possible use of the claimed invention, it does not necessarily limit the claimed invention to having only that purpose, benefit or possible use.

In the present specification, terms such as “part”, “component”, “means”, “section”, or “segment” may refer to singular or plural items and are terms intended to refer to a set of properties, functions, or characteristics performed by one or more items having one or more parts. It is envisaged that where a “part”, “component”, “means”, “section”, “segment”, or similar term is described as consisting of a single item, then a functionally equivalent object consisting of multiple items is considered to fall within the scope of the term; and similarly, where a “part”, “component”, “means”, “section”, “segment”, or similar term is described as consisting of multiple items, a functionally equivalent object consisting of a single item is considered to fall within the scope of the term. The intended interpretation of such terms described in this paragraph should apply unless the contrary is expressly stated or the context requires otherwise.

The term “connected” or a similar term, should not be interpreted as being limited to direct connections only. Thus, the scope of the expression “an item A connected to an item B” should not be limited to items or systems wherein an output of item A is directly connected to an input of item B. It means that there exists a path between an output of A and an input of B which may be a path including other items or means. “Connected”, or a similar term, may mean either that two or more elements are in direct physical or causal contact, or that two or more elements are not in direct contact with each other yet still co-operate or interact with each other.

It will be appreciated by persons skilled in the art that numerous variations or modifications may be made to the present technology as shown in the specific implementations without departing from the spirit or scope of the invention as broadly described. For example, any formulas given above are merely representative of procedures that may be used. Functionality may be added or deleted from the block diagrams and operations may be interchanged among functional blocks. Steps may be added or deleted to methods described within the scope of the present technology. The disclosed implementations are, therefore, to be considered in all respects as illustrative and not limiting or restrictive.

The features described in relation to one or more aspects of the present technology are to be understood as applicable to other aspects of the present technology. More generally, combinations of the steps in the method(s) of the present technology or the features of the system(s) or device(s) of the present technology described elsewhere in the present disclosure, including in the claims, are to be understood as falling within the scope of the present disclosure.

### INDUSTRIAL APPLICABILITY

The arrangements described are applicable to the health care industries.

### EXAMPLES

The following is a non-limiting list of examples in accordance with one or more aspects of the present technology.
Example 1. A neural stimulation system comprising:
   a closed-loop neural stimulation device for controllably delivering neural stimuli to a neural pathway, the device comprising a controller configured to adjust, using a feedback controller comprising one or more loop parameters, a stimulus intensity of the delivered neural stimuli so as to maintain a measured intensity of neural responses evoked by the neural stimuli at or near a target response intensity; and
   one or more processors configured to, individually or in combination:
      receive an INA characteristic vector comprising one or more parameters of a model of instantaneous neural accommodation of the neural pathway; and
      set the one or more loop parameters of the feedback controller of the closed-loop neural stimulation device based on the received INA characteristic vector.
Example 2. The system of example 1, wherein one or more processors are, individually or in combination, configured to set the one or more loop parameters so as to achieve a stable low-pass response.
Example 3. The system of example 2, wherein the feedback controller is a proportional-integral-differential feedback controller and the one or more loop parameters are respective controller gains of the proportional-integral-differential feedback controller.
Example 4. The system of example 3, wherein the one or more processors are, individually or in combination, configured to set the controller gains such that the zeroes of a transfer function of the feedback controller cancel the poles of a transfer function defined by the INA characteristic vector.
Example 5. The system of any one of examples 1 to 4, wherein the wherein the model of INA is a bifurcated model comprising positive-going parameters and negative-going parameters.
Example 6. The system of example 5, wherein the one or more loop parameters are respective controller gains of the feedback controller, the controller gains comprising a positive-going controller gain and a negative-going controller gain.
Example 7. The system of example 6, wherein the one or more processors are, individually or in combination, configured to set the controller gains by:
   setting the positive-going controller gain based on the positive-going parameters of the INA model; and
   setting the negative-going controller gain based on the negative-going parameters of the INA model.
Example 8. A method of programming a closed-loop neural stimulation device comprising a controller configured to adjust, using a feedback controller comprising one or more loop parameters, a stimulus intensity of delivered neural stimuli so as to maintain a measured intensity of neural responses evoked by the neural stimuli from a neural pathway of a patient at or near a target response intensity, the method comprising:
   receive an INA characteristic vector comprising one or more parameters of a model of instantaneous neural accommodation of the neural pathway; and
   set the one or more loop parameters of the feedback controller of the closed-loop neural stimulation device based on the received INA characteristic vector.
Example 9. A closed-loop neural stimulation device for controllably delivering neural stimuli to a neural pathway, the device comprising:
   a controller configured to adjust, using a feedback controller comprising one or more loop parameters, a stimulus intensity of the delivered neural stimuli so as to maintain a measured intensity of neural responses evoked by the neural stimuli at or near a target response intensity,
   wherein the one or more loop parameters have been configured to achieve a stable low-pass response in the presence of instantaneous neural accommodation of the neural pathway.
Example 10. The device of example 9, wherein the feedback controller is a proportional-integral-differential (PID) controller and the one or more loop parameters comprise a proportional gain, an integral gain, and a differential gain.
Example 11. A method of estimating one or more key parameters of a response of target neural fibres to neural stimuli, the target fibres being subject to instantaneous neural accommodation (INA), the method comprising:
   delivering a test sequence of neural stimuli to the neural pathway, each neural stimulus being configured to evoke a neural response from the target neural fibres, each neural stimulus being delivered according to a stimulus intensity parameter;
   capturing a signal window from each signal sensed from the target neural fibres subsequent to the respective neural stimuli;
   measuring intensities of neural responses evoked by the respective neural stimuli in the signal windows; and
   determining, from the stimulus intensity parameters and the respective measured intensities of the evoked neural responses, one or more key parameters of the response of the target neural fibres,
   wherein the test sequence is configured such that the key parameters represent either:
      an accommodated response, or
      an un-accommodated response
   of the target neural fibres.
Example 12. The method of example 11, wherein the test sequence is a sequence of isolated individual stimuli of varying intensity, separated from each other by a time interval that is longer than time period required for excitability of the target neural fibres to return to its baseline state.
Example 13. The method of example 11, wherein the test sequence comprises:
   a plurality of sequences of stimuli at a constant stimulus intensity with a duration much longer than the expected time constant of the INA of the target neural fibres;
   wherein the constant stimulus intensity decreases after each sequence.
Example 14. The method of example 13, where only one or more response intensities at the end of each sequence are used to determine the one or more key parameters.
Example 15. A closed-loop neural stimulation device for controllably delivering neural stimuli to a neural pathway, the device comprising:
   a controller configured to adjust, using a feedback controller, a stimulus intensity of the delivered neural stimuli so as to maintain a property of the neural pathway at or near a target value so as to achieve a more stable sensation of stimulation as posture varies in the presence of instantaneous neural accommodation (INA) of the neural pathway.
Example 16. The device of example 15, wherein the controller configured to adjust, using the feedback controller, a stimulus intensity of the delivered neural stimuli so as to maintain a measured intensity of neural responses evoked by the neural stimuli at or near a target response intensity.
Example 17. The device of example 16, wherein the controller is configured to dynamically adjust the target response intensity based on one or more parameters of a model of the INA.
Example 18. The device of example 16, wherein the controller is configured to further adjust the stimulus intensity based on one or more parameters of a model of the INA.

LABEL LIST

| | | | |
|---|---|---|---|
| stimulator | 100 | ground | 130 |
| patient | 108 | array | 150 |
| electronics module | 110 | biphasic stimulus pulse | 160 |
| battery | 112 | ECAPs | 170 |
| telemetry module | 114 | target fibres | 180 |
| controller | 116 | communications channel | 190 |
| memory | 118 | external computing device | 192 |
| clinical data | 120 | CLNS system | 300 |
| clinical settings | 121 | clinical settings controller | 302 |
| control programs | 122 | target ECAP controller | 304 |
| pulse generator | 124 | box | 308 |
| electrode selection module | 126 | box | 309 |
| measurement circuitry | 128 | feedback controller | 310 |
| box | 311 | INA time series model | 909 |
| pulse generator | 312 | PID controller | 910 |
| summing element | 313 | gain element | 936 |
| measurement circuitry | 318 | integrator | 938 |
| signal window | 319 | gain element | 940 |
| ECAP detector | 320 | gain element | 942 |
| comparator | 324 | differentiator | 944 |
| gain element | 336 | summing element | 946 |
| integrator | 338 | graph | 1000 |
| activation plot | 402 | time series model | 1010 |
| ECAP threshold | 404 | INA parameter | 1012 |
| discomfort threshold | 408 | INA parameter | 1014 |
| perception threshold | 410 | INA parameter | 1016 |
| therapeutic range | 412 | time trace | 1020 |
| target ECAP amplitude | 420 | initial step size | 1022 |
| ECAP | 600 | final step size | 1024 |
| neural stimulation system | 700 | INA parameter | 1026 |
| neuromodulation device | 710 | time trace | 1060 |
| remote controller | 720 | step | 1064 |
| CST | 730 | time constant | 1066 |
| CI | 740 | time trace | 1070 |
| charger | 750 | step | 1074 |
| graph | 800 | time constant | 1076 |
| set | 810 | method | 1100 |
| set | 820 | step | 1110 |
| activation plot | 830 | step | 1120 |
| activation plot | 840 | step | 1130 |
| CLNS system | 900 | step | 1140 |

1. A method of determining one or more parameters of a model of instantaneous neural accommodation (INA) of a neural pathway of a patient, the method comprising:
delivering a test sequence of neural stimuli to the neural pathway of the patient, each neural stimulus in the test sequence being configured to evoke a neural response from the neural pathway, each neural stimulus in the test sequence being delivered according to a respective stimulus intensity parameter;
for each delivered neural stimulus in the test sequence:
capturing at least one signal window from a signal sensed on the neural pathway subsequent to the delivery of that neural stimulus; and
measuring, from the at least one signal window, an intensity of a neural response evoked by that neural stimulus; and
determining, from (i) the respective stimulus intensity parameters and (ii) the measured intensities of the neural responses evoked by the test sequence, first values for the one or more parameters of the model of instantaneous neural accommodation (INA) of the neural pathway.

2. The method of claim 1, wherein determining the first values for the one or more parameters comprises applying a regression model to the respective stimulus intensity parameters and the measured intensities of the neural responses.

3. The method of claim 1, wherein the model of INA is an autoregressive moving average (ARMA) model.

4. The method of claim 1, wherein the test sequence comprises a step of the stimulus intensity parameters.

5. The method of claim 1, wherein the model of INA is a bifurcated model, and wherein the one or more parameters comprise positive-going parameters and negative-going parameters.

6. The method of claim 5,
wherein the test sequence comprises stepped-up stimulus intensity parameters, and
wherein determining the first values for the one or more parameters of the model of INA of the neural pathway comprises determining, from (i) the stepped-up stimulus intensity parameters and (ii) the measured intensities of the neural responses evoked by the test sequence, first values for the positive-going parameters.

7. The method of claim 5,
wherein the test sequence comprises stepped-down stimulus intensity parameters, and
wherein determining the first values for the one or more parameters of the model of INA of the neural pathway comprises determining, from (i) the stepped-down stimulus intensity parameters and (ii) the measured intensities of the neural responses evoked by the test sequence, first values for the negative-going parameters.

8. The method of claim 1, further comprising classifying the patient into one of a plurality of categories based on the first values for the parameters of the model of INA.

9. The method of claim 8, wherein the plurality of categories comprises responders to neural stimulation therapy, and non-responders to neural stimulation therapy.

10. The method of claim 1, further comprising predicting one or more characteristics of a patient's response to neural stimulation therapy based on the first values for the parameters of the model of INA.

11. The method of claim 1, further comprising:
repeating the delivering, capturing, measuring, and determining further values for the one or more parameters of the model of INA, and
monitoring a progression of a condition of the patient based on the first values and the further values for the one or more of the parameters of the model of INA.

12. The method of claim 1, further comprising programming a neural stimulation system to deliver therapeutic neural stimuli to the neural pathway based on the first values for the one or more parameters.

13. A neural stimulation system comprising:
an implantable device comprising:
a pulse generator configured to deliver neural stimuli to a neural pathway of a patient, the neural stimuli being configured to evoke neural responses from the neural pathway;
measurement circuitry configured to capture signal windows from signals sensed on the neural pathway by one or more measurement electrodes; and
a controller configured to:
control the pulse generator to deliver each neural stimulus; and
control the measurement circuitry to capture a signal window from a signal sensed on the neural pathway subsequent to each neural stimulus; and
a test module comprising a processor, the test module configured to:
cause the controller to control the pulse generator to deliver a test sequence of neural stimuli, each neural stimulus of the test sequence being delivered according to a respective stimulus intensity parameter;
cause the controller to control the measurement circuitry to capture a signal window from a signal sensed on the neural pathway subsequent to each neural stimulus of the test sequence;
for each delivered neural stimulus of the test sequence, measure an intensity of a neural response evoked by that neural stimulus from a signal window captured subsequent to the delivery of that neural stimulus; and
determine, from i) the respective stimulus intensity parameters of the test sequence and ii) the measured intensities of the neural responses evoked by the test sequence, first values for one or more parameters of a model of instantaneous neural accommodation (INA) of the neural pathway.

14. The neural stimulation system of claim 13, wherein determining the first values for the one or more parameters comprises applying a regression model to the stimulus intensity parameters and the measured intensities of the neural responses.

15. The neural stimulation system of claim 13, wherein the model of INA is an autoregressive moving average (ARMA) model.

16. The neural stimulation system of claim 13, wherein the test sequence comprises a step of the stimulus intensity parameters.

17. The neural stimulation system of claim 13, wherein the model of INA is a bifurcated model, and wherein the one or more parameters comprise positive-going parameters and negative-going parameters.

18. The neural stimulation system of claim 17,
wherein the test sequence comprises stepped-up stimulus intensity parameters, and
wherein determining the first values for the one or more parameters of the model of INA of the neural pathway comprises determining, from (i) the stepped-up stimulus intensity parameters and (ii) the measured intensities of the neural responses evoked by the test sequence, first values for the positive-going parameters.

19. The neural stimulation system of claim 17,
wherein the test sequence comprises stepped-down stimulus intensity parameters, and
wherein determining the first values for the one or more parameters of the model of INA of the neural pathway comprises determining, from (i) the stepped-down stimulus intensity parameters and (ii) the measured intensities of the neural responses evoked by the test sequence, first values for the negative-going parameters.

20. The neural stimulation system of claim 13, wherein the controller is configured to deliver therapeutic neural stimuli to the neural pathway based on the first values for the one or more parameters.
